(19) United States
(12) Patent Application Publication
(10) Pub. No.: US 2011/0029077 A1
(43) Pub. Date: Feb. 3, 2011

(54) MEDICAL IMPLANT

(76) Inventor: Jong-Soo Choi, Seoul (KR)

Correspondence Address: HARNESS, DICKEY & PIERCE, P.L.C., P.O. BOX 8910, RESTON, VA, 20195, US

(21) Appl. No.: 12/921,244

(22) PCT Filed: Mar. 12, 2008
(86) PCT No.: PCT/KR2008/001391

§ 371 Date: Sep. 7, 2010

(51) Int. Cl. A61F 2/12

(52) U.S. Cl. 623/8

(57) ABSTRACT

A medical implant to be accommodated in a human body is disclosed. The medical implant includes porous silicon with bubbles of different sizes, and a silicon film enclosing the porous silicon. Also, the medical implant includes porous silicon balls with bubbles and a silicon film enclosing the porous silicon balls. Cushion and weight of the implant can be properly adjusted so as to maintain the cushion at a level similar to that of cellular cells of the human body and to shorten a recovery time. Plural grooves or through-holes are provided on a surface of the implant, and thus body fluid smoothly flows through the grooves or through-holes to improve the affinity of the implant against the cellular cells.

### TECHNICAL FIELD

The present invention relates to a medical implant to be accommodated in a human body, in which the implant is composed of porous silicon with bubbles of different sizes, and a silicon film enclosing the porous silicon. Also, the present invention relates to a medical implant including through-holes penetrating the implant in a vertical direction and a plurality of valleys formed on a surface of the implant.

### BACKGROUND ART

Generally, implants are used to restore a depression or specific portion of a human body, as well as performing operation of breast enlargement of women or corpus cavernosum of penis. In particular, such an implant is generally made of silicon material which is not harmful to a human body, so as to lessen rejection symptom or resistance against a surgical site. A silicon bag filled with a physiological slat solution or solid silicon is used depending upon a surgical area of the human body.

### DISCLOSURE

### Technical Problem

There is a drawback that the silicon bag implant is heavy, while the shape thereof is not maintained properly. In particular, it should pay close attention to prevent damage of the silicon bag from being damaged during the surgical operation. Also, if the silicon bag implant is damaged or burst during or after the surgical operation, drain of a filling material give rise to side effects on the human body.

One example of the solid silicon implant is disclosed in Korean Utility Model Unexamined Publication No. 1996-033540, the implant having a plurality of through-holes, through which a body fluid flows. Because of the lowered sensitivity in tactile sensation or elasticity, a person surgically operated feels the resistance or strangeness. Also, because of the lowered affinity with granulation tissues, the solid silicon implant is not fixed in the granulation tissues and thus is moved therein.

Therefore, the present invention has been made to solve the above-mentioned problems occurring in the prior art, and an object of the present invention is to provide a medical implant includes porous silicon with bubbles of different sizes, and a silicon film enclosing the porous silicon.

### Technical Solution

In order to accomplish the above-mentioned objects, the present invention provides a medical implant including porous silicon with bubbles or a porous silicon structure of a double construction. The medical implant includes porous silicon with bubbles therein, and a silicon film enclosing the porous silicon, a plurality of valleys being formed on an outer surface of the silicon film.

Also, the medical implant of the present invention is used to restore or enlarge a depression or specific portion of a human body, in which a plurality of through-holes are formed on the surface of the implant, and a plurality of vertical and horizontal valleys are formed on its surface, through which the human fluid flows. It can increase the affinity between the implant and the body tissue and thus prevent the implant from being released from a surgical site.

### ADVANTAGEOUS EFFECTS

With the above construction, the medical implant of the present invention can prevent the silicon from flowing in the human body even though the silicon film is damaged in the human body, since the implant has a solid silicon construction. Since the implant can be lighter, if necessary, it can solve the difficulty of a patient due to excessive weight. Also, when the implant with the valleys formed on its surface is introduced in the human body, the body fluid flows along the valleys to improve the affinity between the implant and the body tissue. In case the through-holes are formed, the affinity is further increased. In addition, the implant having the double construction can be manufactured to have different hardness and material, so that the implant can be suitable for each portion of the human body to increase the affinity between the implant and the body tissue.

### DESCRIPTION OF DRAWINGS

FIG. 1 is a cross-sectional view illustrating a medical implant according to a first embodiment of the present invention;

FIG. 2 is a cross-sectional view illustrating a medical implant according to a second embodiment of the present invention;

FIG. 3 is a plan view illustrating a medical implant according to a second embodiment of the present invention;

FIG. 4 is a perspective view illustrating a medical implant according to a second embodiment of the present invention;

FIG. 5 is a cross-sectional view illustrating a medical implant according to a third embodiment of the present invention;

FIG. 6 is a cross-sectional view illustrating a medical implant according to a fourth embodiment of the present invention;

FIG. 7 is a plan view illustrating a medical implant according to a fifth embodiment of the present invention;

FIG. 8 is a cross-sectional view illustrating a medical implant according to a fifth embodiment of the present invention; and

FIG. 9 is a cross-sectional view illustrating a medical implant according to a sixth embodiment of the present invention.

### MODE FOR INVENTION

Hereinafter, preferred embodiments of the present invention will be described in detail with reference to the accompanying drawings. In the whole description of the present invention, the same drawing reference numerals are used for the same elements among/across various figures.

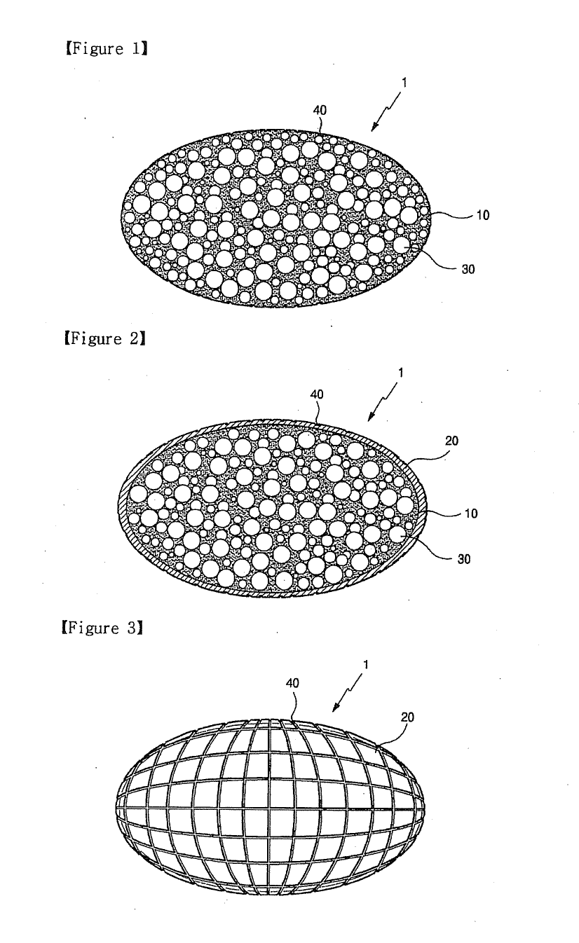

Referring to FIG. 1 which is a cross-sectional view illustrating a medical implant according to a first embodiment of the present invention, the implant is made of porous silicon with bubbles therein. When bubbles 30 contained in the porous silicon are exposed from the surface of the porous silicon, grooves are formed on the surface, and thus a body fluid stays in the grooves. In order to prevent the stay of body fluid, valleys 40 are formed on the surface so that the body fluid flows along the valleys 40 to prevent the stay of the body fluid. Since the valleys 40 are formed on the surface and additional silicon films are not necessarily formed, the implant according to the first embodiment can be easily manufactured as compared with other embodiments in which the implant includes double construction composed of a porous silicon 10 and a silicon film 20. The implant according to the first embodiment includes the steps of: selecting materials for manufacturing the porous silicon 10 and putting a silicon material into a furnace; heating and melting the silicon material put into the furnace; gradually cooling the molten silicon material, while stirring the molten silicon material with a bar to generate bubbles therein; and introducing the molten silicon material with the bubbles into a mold. In the forming step, the valleys 40 for formed on the surface of the medical implant.

Referring to FIG. 2 which is a cross-sectional view illustrating a medical implant according to a second embodiment of the present invention, the implant 1 includes porous silicon 10 with bubbles 30 of different sizes, and a silicon film 20 enclosing the porous silicon 10. The porous silicon 10 serves as an internal filler in the construction of the present invention to adjust an amount of the bubbles formed in the porous silicon 10, thereby controlling its weight and hardness. The method of forming the bubbles will be described hereinafter. The silicon film 20 serves as a sheath, and is preferably made of a material having hardness higher than that of the porous silicon 10.

Referring to FIG. 3 which is a plan view illustrating a medical implant according to a second embodiment of the present invention, the implant further includes valleys 40 formed on the surface of the silicon film 20. In this instance, the valleys 40 may be closely formed on the entire outer surface of the silicon film 20. The valleys 40 may have horizontal valleys 40a and vertical valleys 40b crossing the horizontal valleys 40a. Since the body fluid naturally flows along the valleys 40, it can prevent an adverse effect resulted from the staying of body fluid which is a problem of the prior art.

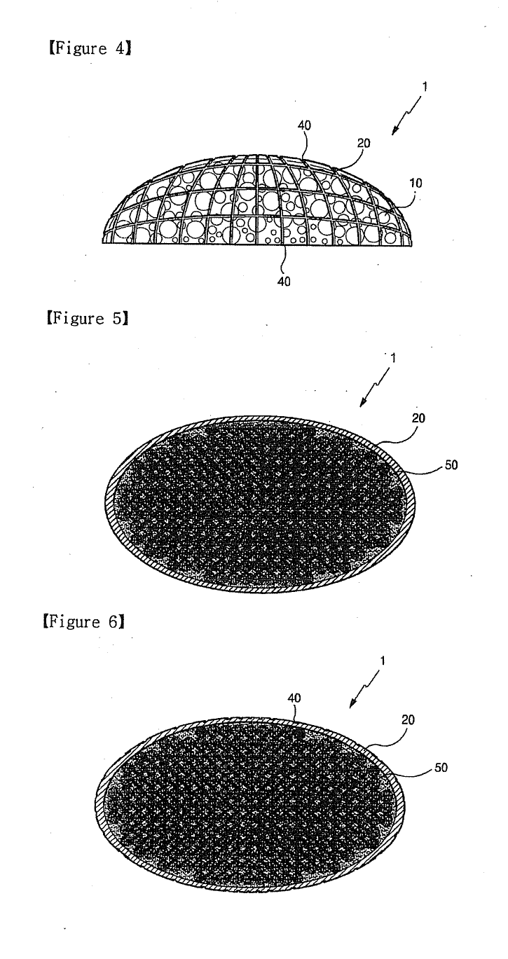

Referring to FIG. 4 which is a perspective view illustrating a medical implant according to a second embodiment of the present invention, the implant has a double construction composed of porous silicon 10 and silicon layer 20.

The implant according to the second embodiment includes the steps of: forming the porous silicon 10 with bubbles 30 having different size; and the silicon film 20 enclosing the porous silicon 10. The method may further include forming valleys 40 on the entire outer surface of the silicon film 20. The step of forming the porous silicon 10 includes the steps of: selecting a silicon material suitable for hardness according to surgical operation, and putting the selected silicon material into a furnace; heating and melting the silicon material put into the furnace; gradually cooling the molten silicon material, while stirring the molten silicon material with a bar to generate bubbles therein; and introducing the molten silicon material with the bubbles into a mold to shape the implant. The mold of the porous silicon 10 may be made by a mold which is formed in various shapes according to an object to perform surgical operation.

After the material of the porous silicon 10 is selected according to needed hardness, the material is put into the furnace, and then is heated by a temperature of about 200° C. to 280° C. to melt the material. Next, the molten material is gradually cooled, while the molten material is manually or automatically quickly stirred by using the rod. The reason why the molten material is stirred is that the bubbles 30 are formed in the porous silicon. This is to reduce a weight of the implant and improve the natural tactile sensation. The porous silicon may be filled with separate gas, such as oxygen, carbon dioxide, or nitrogen, to generate the bubbles. Wanted amounts of bubbles 30 to be generated can be obtained by adjusting stirring speed and time. It should be noted that the amount of bubbles is generally increased in proportion to the stirring time, but its quality may be deteriorated. When the bubbles of a predetermined amount are generated, the molten material is introduced into the mold previously prepared to conduct the forming operation. If the forming is completed after about 10 minutes, the molding is drawn out from the mold, and then is cooled, thereby forming the porous silicon 10.

The step of forming the silicon film 20 includes the steps of: selecting a silicon material suitable for hardness according to surgical operation, and putting the selected silicon material into a furnace; heating and melting the silicon material put into the furnace; introducing the porous silicon 10 into a silicon film mold; and introducing the molten silicon material into the silicon film mold with high pressure. More specifically, a mold for the silicon film 20 is firstly prepared. Preferably, the valleys 40 to be formed on the outer surface of the silicon film 20 are previously formed on an inner periphery of the silicon film mold. After the material of the silicon film is selected according to needed hardness, the material is put into the furnace, and then is heated by a temperature of about 200° C. to 280° C. to melt the material. The prepared porous silicon 10 is introduced into the silicon film mold, and then the molten material is introduced into the silicon film mold with the high pressure. In this instance, the molten material is introduced into the porous silicon 10 and the silicon film mold to form the implant having the double construction composed of the porous silicon 10 and the silicon film 20, as shown in FIG. 2. If the forming is completed after about 10 minutes, the implant is drawn out from the mold, and then is cooled, thereby manufacturing the implant 1.

FIG. 5 is a cross-sectional view illustrating a medical implant according to a third embodiment of the present invention, and FIG. 6 is a cross-sectional view illustrating a medical implant according to a fourth embodiment of the present invention. These embodiments utilize porous silicon balls 50, differently from the porous silicon 10 in the second embodiment. The porous silicon ball 50 has bubbles 30 therein, like the porous silicon 10. The balls have 1 to 100 mm in diameter. That is, according to these embodiments, the liquidity of the implant is improved by filling the silicon film 20 with a plurality of porous silicon balls 50, so that a patient or use can feels the tactile sensation as an actual skin. In FIG. 6, the valleys 40 are formed on the entire surface of the silicon film 20 of the medical implant 1 according to the third embodiment.

The method of manufacturing the medical implant according to this embodiment includes forming a plurality of porous silicon balls 50 with the bubbles 30 having the different size and forming the silicon film 20 enclosing the porous silicon balls 50. The method may further include a step of forming the valleys 40 on the entire surface of the silicon film 20.

The step of forming a plurality of porous silicon balls 50 with the bubbles 30 having different size includes the steps of: selecting a silicon material suitable for the needed hardness, and putting the selected silicon material into a furnace; heating and melting the silicon material put into the furnace; gradually cooling the molten silicon material, while stirring the molten silicon material with a rod to generate bubbles 30 therein; and introducing the molten silicon material with the bubbles 30 into the mold to form the porous silicon balls. The step is similar to the step of forming the porous silicon 10 described above. More specifically, after the material of the porous silicon ball 50 is selected according to needed hardness, the material is put into the furnace, and then is heated by a temperature of about 200° C. to 280° C. to melt the material. The molten material is gradually cooled, and is quickly stirred. The reason why the molten material is stirred is that the bubbles 30 are formed in the porous silicon balls 50. When a necessary amount of the bubbles are generated, the material is put into the mold for the porous silicon ball previously prepared to form the porous silicon ball. If the forming is completed, the porous silicon ball is drawn out from the mold, and then is cooled, thereby forming the porous silicon ball 50.

The step of forming the silicon film 20 includes the steps of: selecting a silicon material suitable for the needed hardness, and putting the selected silicon material into a furnace; heating and melting the silicon material put into the furnace; and introducing the molten silicon material into a silicon film mold with high pressure. The valleys 40 to be formed on the outer surface of the silicon film may bee previously formed on an inner periphery of the silicon film mold. Preferably, the silicon film 20 is introduced into a surgical site of the human body, and then an inlet port is formed on the silicon film 20 by using a separate tube, so that the porous silicon balls 50 are put into the silicon film 20. It can reduce a cut region of the breast or buttocks of a patient to make a quick recovery.

FIG. 7 is a plan view illustrating a medical implant according to a fifth embodiment of the present invention. The medical implant includes a plurality of horizontal valleys 40a and a plurality of vertical valleys 40b which are formed on the surface of the implant 1, and a plurality of through-holes 60 penetrating crossing points which are formed by the horizontal and vertical valleys crossing each other. The weight of the medial implant can be reduced by the valleys 40 and the through-holes 60 formed on the surface. The through-holes 60 are formed at the crossing points of the horizontal and vertical valleys 40a and 40b formed on the surface of the medical implant 1, but may be formed at unspecified positions, regardless of the positions of the valleys 40. If the through-holes 60 are formed at the crossing points of the valleys 40a and 40b, the body fluid generated from the surgical site flows along the valleys, and simultaneously flows via the through-holes 60. Also, since the thickness of the medical implant 1 is varied depending upon its position, depths of the valleys may be formed differently according to the variation of the thickness.

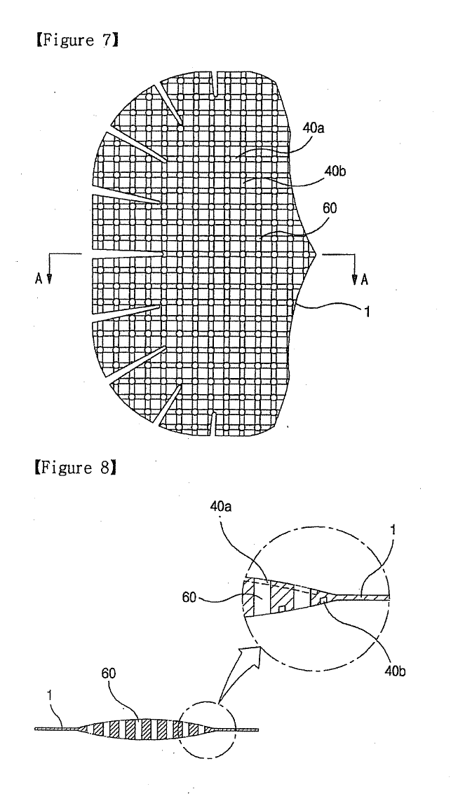

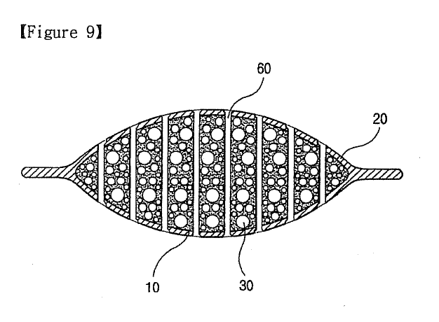

FIG. 8 is a cross-sectional view taken along line A-A of the medical implant shown in FIG. 7, in which a partially enlarged view is also shown. It would be understood that the through-holes 60 penetrate the medical implant 1 in a vertical direction, and the horizontal valley 40a and the vertical valley 40b are formed on the upper and lower surfaces of the medical implant 1. When the medical implant is introduced into the surgical site, the body fluid can freely flow along the valleys and the through-holes in all directions. Also, body tissues may be fixed by the valleys 40 and the through-holes 60 to prevent the medical implant 1 from being released from the surgical site. FIG. 9 is a cross-sectional view illustrating a medical implant according to a sixth embodiment of the present invention. Through-holes 60 are additionally formed on the medical implant according to the second embodiment. Since the through-holes 60 are formed on the medical implant 1 having a double construction consisting of the porous silicon 10 and the silicon film 20, the body fluid can freely flow from one surface to the opposite surface.

After the medical implant of the present invention is introduced into the breast or buttocks, the implant can be returned to its original shape although pressure is applied to the silicon implant from the exterior.

The forgoing embodiments are merely exemplary and are not to be construed as limiting the present invention. The present teachings can be readily applied to other types of apparatuses. The description of the present invention is intended to be illustrative, and not to limit the scope of the claims. Many alternatives, modifications, and variations will be apparent to those skilled in the art.

### INDUSTRIAL APPLICABILITY

As apparent from the above description, the medical implant of the present invention has the double construction composed of the porous silicon and the silicon film, the silicon is not leaked into the human body, unlike a solid silicon. Also, the medical implant has light and good wearing sensation. Since the body fluid smoothly flows along the valleys formed on the surface, the affinity between the implant and the body tissue to make a quick recovery. In addition, since the medical implant has the double construction consisting of the porous silicon and the silicon film or the double construction consisting of the porous silicon balls and the silicon film, the hardness of the implant can be easily adjusted according to the surgical site, thereby increasing the satisfaction after the surgical operation.

Since the medical implant is properly fixed to the body tissue of the surgical tissue by the valleys and the through-holes, the possibility of the implant is decreased from carelessness of the patient after the surgical operation, thereby lowering a re-operation rate.

1. A medical implant to be accommodated in a human body; comprising:
porous silicon with bubbles therein; and
a plurality of valleys formed on a surface of the implant.

2. A medical implant to be accommodated in a human body, comprising:
porous silicon with bubbles therein; and
a silicon film enclosing the porous silicon, a plurality of valleys being formed on an outer surface of the silicon film.

3. A medical implant to be accommodated in a human body, comprising:
a plurality of porous silicon balls with bubbles therein; and
a silicon film enclosing the porous silicon balls.

4. The medical implant of claim 3, wherein a plurality of valleys are formed on an outer surface of the silicon film.

5. The medical implant of claim 1, wherein a plurality of through-holes penetrate the implant in a vertical direction.

6. The medical implant of claim 2, wherein a plurality of through-holes penetrate the implant in a vertical direction.
